(19) United States
(12) Patent Application Publication
(10) Pub. No.: US 2019/0247197 A1
(43) Pub. Date: Aug. 15, 2019

(54) DUAL POSITION CAGE SYSTEMS AND METHODS

(71) Applicant: CTL Medical Corporation, Addison, TX (US)

(72) Inventors: Jayant JAGANNATHAN, Troy, MI (US); Sidd SINGH, Ann Arbor, MI (US); Jon SUH, Ambler, PA (US); Sean SUH, Milltown, NJ (US)

(21) Appl. No.: 16/271,095

(22) Filed: Feb. 8, 2019

Related U.S. Application Data
(60) Provisional application No. 62/628,716, filed on Feb. 9, 2018.

(51) Int. Cl. A61F 2/44

(52) U.S. Cl. CPC A61F 2/447; A61F 2002/30153

(57) ABSTRACT

Disclosed are devices, methods and/or systems for intervertebral and intradiscal implants deployed within the body of a patient, including improved interbody devices having internal and/or external characteristics that facilitate use of the implant in a plurality of different orientations.

### CROSS-REFERENCE TO RELATED APPLICATIONS

This application claims the benefit of U.S. Provisional Patent Application Ser. No. 62/628,716 entitled “DUAL POSITION TLIF CAGE,” filed Feb. 9, 2018, the disclosure of which is incorporated by reference herein in its entirety.

### TECHNICAL FIELD

The invention relates generally to medical devices, and more specifically relates to intervertebral and intradiscal systems, devices and surgical methods for deployment within the body of a patient.

### BACKGROUND OF THE INVENTION

In mammals, the spinal (or vertebral) column is one of the most important parts. The spinal column provides the main support necessary for mammals to stand, bend, and twist.

In humans, the spinal column is generally formed by individual interlocking vertebrae, which are classified into five segments, including (from head to tail) a cervical segment (vertebrae C1-C7), a thoracic segment (vertebrae T1-T12), a lumbar segment (vertebrae L1-L5), a sacrum segment (vertebrae S1-S5), and coccyx segment (vertebrate Co1-Co5). The cervical segment forms the neck, supports the head and neck, and allows for nodding, shaking and other movements of the head. The thoracic segment attaches to ribs to form the ribcage. The lumbar segment carries most of the weight of the upper body and provides a stable center of gravity during movement. The sacrum and coccyx make up the back walls of the pelvis.

Intervertebral discs are located between each of the movable vertebra. Each intervertebral disc typically includes a thick outer layer called the disc annulus, which includes a crisscrossing fibrous structure, and a disc nucleus, which is a soft gel-like structure located at the center of the disc. The intervertebral discs function to absorb force and allow for pivotal movement of adjacent vertebra with respect to each other.

In the vertebral column, the vertebrae increase in size as they progress from the cervical segment to the sacrum segment, becoming smaller in the coccyx. At maturity, the five sacral vertebrae typically fuse into one large bone, the sacrum, with no intervertebral discs. The last three to five coccygeal vertebrae (typically four) form the coccyx (or tailbone). Like the sacrum, the coccyx does not have any intervertebral discs.

Each vertebra is an irregular bone that varies in size according to its placement in the spinal column, spinal loading, posture and pathology. While the basic configuration of vertebrae varies, every vertebra has a body that consists of a large anterior middle portion called the centrum and a posterior vertebral arch called the neural arch. The upper and lower surfaces of the vertebra body give attachment to intervertebral discs. The posterior part of a vertebra forms a vertebral arch that typically consists of two pedicles, two laminae, and seven processes. The laminae give attachment to the ligament flava, and the pedicles have a shape that forms vertebral notches to form the intervertebral foramina when the vertebrae articulate. The foramina are the entry and exit passageways for spinal nerves. The body of the vertebra and the vertical arch form the vertebral foramen, which is a large, central opening that accommodates the spinal canal that encloses and protects the spinal cord.

The body of each vertebra is composed of cancellous bone that is covered by a thin coating of cortical bone. The cancellous bone is a spongy type of osseous tissue, and the cortical bone is a hard and dense type of osseous tissue. The vertebral arch and processes have thicker coverings of cortical bone.

The upper and lower surfaces of the vertebra body are flattened and rough. These surfaces are the vertebral endplates that are in direct contact with the intervertebral discs. The endplates are formed from a thickened layer of cancellous bone, with the top layer being denser. The endplates contain adjacent discs and evenly spread applied loads. The endplates also provide anchorage for the collagen fibers of the disc.

In a typical spinal column, each disc forms a fibrocartilaginous joint between adjacent vertebrae so as to allow relative movement between adjacent vertebrae. Beyond enabling relative motion between adjacent vertebrae, each disc also acts as a shock absorber for the spinal column. Each disc comprises a fibrous exterior surrounding an inner gel-like center which cooperate to distribute pressure evenly across each disc, thereby preventing the development of stress concentrations that might otherwise damage and/or impair vertebrae of the spinal column. The discs are, however, subject to various injuries and/or disorders which may interfere with a disc's ability to adequately distribute pressure and protect vertebrae. For example, disc herniation, degeneration, and infection of discs may result in insufficient disc thickness and/or support to absorb and/or distribute forces imparted to the spinal column. Disc degeneration, for example, may result when the inner gel-like center begins to dehydrate, which may result in a degenerated disc having decreased thickness. This decreased thickness may limit the ability of a degenerated disc to absorb shock which, if left untreated, may result in pain and/or vertebral injury.

While pain medication, physical therapy, and other non-operative conditions may alleviate some symptoms, such interventions may not be sufficient for every patient. Accordingly, various procedures have been developed to surgically improve patient quality of life via abatement of pain and/or discomfort. Such procedures may include, discectomy and fusion procedures, such as, for example, anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF). During a discectomy, all or a portion of a damaged disc is removed via an incision, typically under X-ray guidance.

Following the discectomy procedure, a medical professional may determine an appropriate size of a surgical implant such as an interbody device via one or more distractors and/or trials of various sizes. Each trial and/or distractor may be forcibly inserted between adjacent vertebrae. Upon determination of an appropriate size, one or more of an ACIF, ALIF, DLIF, PLIF, and/or TLIF may be performed by placing an appropriate interbody device (such as, for example, a cage, a spacer, or a block) between adjacent vertebrae in the space formed by the removed degenerated disc. Placement of such interbody devices within one or more treated levels of a spinal column may prevent spaces between adjacent vertebrae from collapsing, thereby preventing adjacent vertebrae from resting immediately on top of one another and inducing fracture of vertebra, impingement of the spinal cord, and/or pain. Additionally, such interbody devices may facilitate fusion between adjacent vertebrae by stabilizing adjacent vertebrae relative to one another. In order to facilitate stability of the treated spinal level(s), such interbody devices often may be used in conjunction with one or more bone screws, stabilization plates and/or other hardware, some of which may be attached to and/or extending through the interbody device and/or adjacent vertebrae.

In most cases the sizes and/or shapes of the appropriate interbody devices are only determined in-situ, often because the full extent of anatomical degeneration and/or the required amount of bone removal and/or decompression only become apparent during the actual surgical procedure. As such, a large number of alternative sizes, shapes and/or types of implants are often prepared, sterilized and made available for use in the surgical theatre, most of which remain unused and must be repackaged and/or reprocessed for use in future surgical procedures. This represents a significant investment in inventory, cost and worker effort, and thus there remains a need for improved interbody devices, associated systems, and methodologies related thereto.

### BRIEF SUMMARY OF THE INVENTION

The invention disclosed herein includes the realization of a need for improved interbody devices having features capable of adapting to a plurality of sizes, shapes and/or other aspects of an intended anatomical implant site during a spinal surgical procedure. Such adaptation features will desirably allow for a greatly reduced number of implant components necessary for a given surgical procedure, resulting in a significant reduction in the time, cost and/or expenses associated with preparation and/or use of the surgical implants.

In some embodiments, the improved interbody devices can comprise a surgical cage implant having internal and/or external characteristics that facilitate use of the cage implant in a plurality of different orientations. For example, the surgical cage implant may have a first height at a first orientation and a second, different height at a second orientation, with the surface and/or internal features of the cage allowing for implantation of the cage in the first or second orientations at the surgeon's option. Desirably, the surface features of the cage will provide for implant stability at each of the plurality of orientations and/or positions.

In at least one embodiment, the improved interbody devices can include features that facilitate visualization of the implant and/or of the surrounding anatomy after implantation of the device. For example, the implant can desirably include openings or windows that can accommodate bone graft or other materials to desirably promote healing and/or fusion of the vertebral level. In many cases, the locations, orientations and/or designs of the windows will allow unimpeded visualization through the implant in multiple orientations, thereby allowing a surgeon to determine proper implant placement and/or alignment as well as proper progression of bone arthrodesis.

In various embodiments, the improved interbody devices will desirably comprise single-piece implants with no moving components, while in other embodiments multi-piece, modular and/or adjustable components may be utilized that incorporate various of the advantages described herein.

If desired, the various improved interbody devices described herein could be provided in a kit form, with a plurality of such interbody devices of differing shapes, sizes and/or other features.

Additional features, advantages, and embodiments of the disclosure may be set forth or apparent from consideration of the detailed description and drawings. Moreover, it is to be understood that both the foregoing summary of the disclosure and the following detailed description are exemplary and intended to provide further explanation without limiting the scope of the disclosure as claimed.

### BRIEF DESCRIPTION OF THE SEVERAL VIEWS OF THE DRAWINGS

FIG. 1 depicts a perspective view of one exemplary embodiment of a dual-position spinal cage implant;

FIG. 2 depicts an additional perspective view of the of the dual-position spinal cage implant of FIG. 1;

FIGS. 3A through 3C depict side views of a dual-position implant in Primary and Secondary alignment positions;

FIG. 4 depicts a cross-section view of the dual-position spinal cage implant of FIG. 2; and

FIGS. 5A through 5E depict various views of the dual-position spinal cage implant of FIG. 1.

### DETAILED DESCRIPTION OF THE INVENTION

The disclosures of the various embodiments described herein are provided with sufficient specificity to meet statutory requirements, but these descriptions are not necessarily intended to limit the scope of the claims. The claimed subject matter may be embodied in a wide variety of other ways, may include different steps or elements, and may be used in conjunction with other technologies, including past, present and/or future developments. The descriptions provided herein should not be interpreted as implying any particular order or arrangement among or between various steps or elements except when the order of individual steps or arrangement of elements is explicitly described.

Various embodiments described herein include the design and manufacture of improved interbody devices having features capable of adapting to a plurality of sizes, shapes and/or other aspects of an intended anatomical implant site during a spinal surgical procedure. In various embodiments, this can include features that facilitate use of an interbody device at differing orientations, with each orientation of the implant allowing the implant to accommodate a different size, shape and/or other aspect of a targeted anatomical location.

Disclosed herein are systems, devices, methods and surgical procedures for implants that can be utilized at a plurality of orientations, with each orientation particularly suited to accommodate the shape and/or size of particular patient anatomy. In various embodiments, the implant devices can include a first orientation that presents a first height, width, length and/or surface shape/texture to the patient's anatomy (such as, for example, the upper and lower endplates of a patient's adjacent vertebral bodies), and a second orientation that presents a second height, width, length and/or surface shape/texture to the patient anatomy, with the first height, width, length and/or surface shape/texture being different from the second height, width, length and/or surface shape/texture. Desirably, this would allow a single implant to be utilized in a plurality of surgical situations for the same and/or different patients, potentially reducing the number of implant sizes, shapes and/or other component combinations needed to accomplish a given surgery.

While dual or two-position cages are specifically disclosed herein, various alternative embodiments could include three or four-position designs, where the opposing surfaces (i.e., upper and lower) could be of differing sizes and/or shapes, etc., if desired. Similarly, additional functional surfaces could be provided on the ends of the implant, leading to the potential for five or six-position implants, if desired.

In various embodiments, an intervertebral fusion cage is disclosed, the cage desirably incorporating dual-position footprint pairs where either the superior and inferior surface pair (primary orientation) or the medial and lateral surface pair (secondary orientation) can be placed into intimate contact with the vertebral bodies above and below a targeted anatomical disc region in which the implant is installed during a surgical procedure. In one exemplary embodiment, the implant will desirably include similar surface features for both the primary and secondary pairs, and will further desirably include openings, windows and/or other features that greatly facilitate use of the implant in either the primary or secondary orientation, including graft placement and visualization windows allowing post-surgery fluoroscopic visualization of the implant and targeted anatomy after the implant has been installed on the vertebral body disc space to maintain a normal disc height. Both of the surface pairs can have geometric textures that desirably engage with the upper and/or lower endplates to resist expulsion from the targeted anatomy, and the implant further can include through window(s) and/or recessed pockets for accommodating bone growth inducing and/or conducting and/or stimulating agent(s) such as bone graft materials and/or blood, etc.

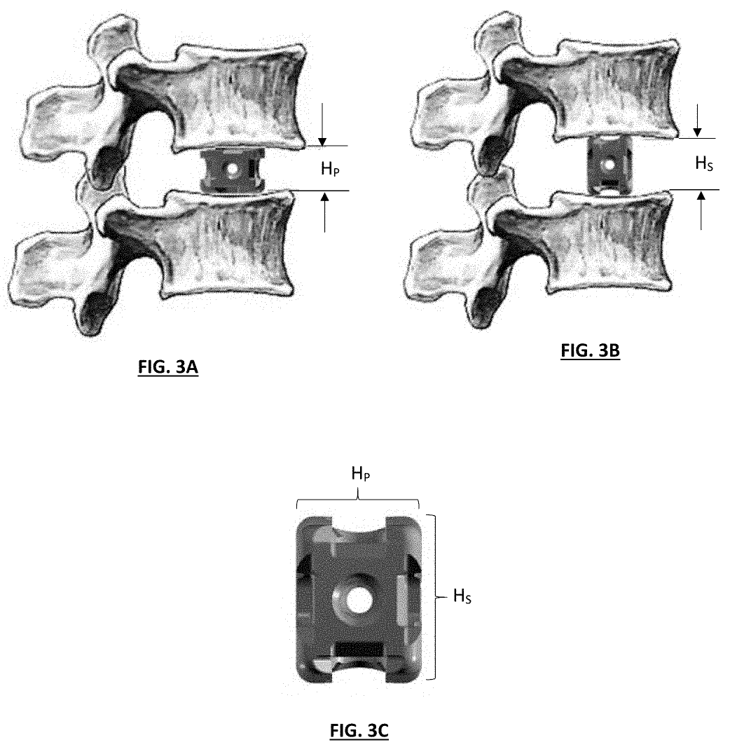

In various embodiments, the disclosed implants can desirably help minimize inventory requirements for a given surgical procedure by providing a plurality of sizes in a single cage implant. Following a discectomy or other procedure, a medical professional may determine an appropriate size of the cage by selecting an appropriately dimensioned cage which may be selectable based on, for example, height, width, depth, number of graft chambers, configuration of graft chambers, configuration of outer surface(s), and the like. Upon selecting the appropriate cage, one or more of an ACIF, ALIF, or the like may be performed by placing the cage between adjacent vertebrae in a space formed by the removed degenerated disc (see FIGS. 3A and 3B). Placement of the cage within the spinal column will desirably prevent spaces between adjacent vertebrae from collapsing, thereby preventing adjacent vertebrae from resting immediately on top of one another and inducing fracture of the vertebra, impingement of the spinal cord, and/or pain. Additionally, the cage will desirably facilitate fusion (e.g., bone to grow together) between adjacent vertebrae by stabilizing the adjacent vertebrae relative to one another.

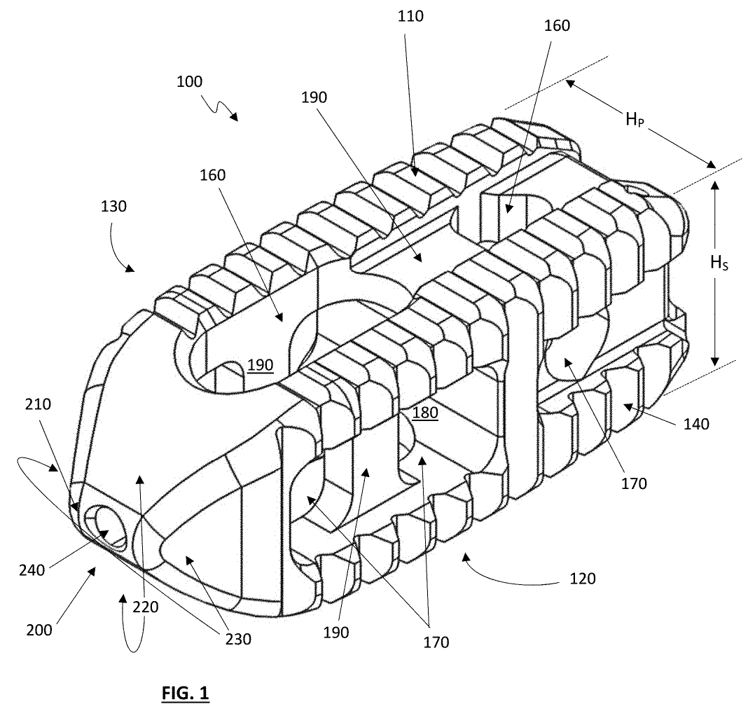

FIG. 1 depicts a perspective view of one exemplary embodiment of a dual-position cage 100, which includes a cage body 110 with a superior surface 120, and inferior surface 130, a medial surface 140 and a lateral surface 150. Desirably, each of these surfaces 110, 120, 130 and 140 include surface features appropriate for placement against the upper and/or lower vertebral bodies of a targeted anatomical region of the spine, which can include ribbed and/or textured surfaces as well known in the art. The cage body also desirably includes one or more vertical windows or openings 160, along with one or more horizontal windows or openings 170, with some or all of the vertical and horizontal windows desirably in fluid communication with a central void 180 within the cage body 110. The cage body 110 can further includes vertical and horizontal support bars 190, which desirably provide additional strength and support for the cage against compressive loading within the targeted anatomy. In various embodiments, some or all of the support bars 190 will be recessed into the cage body 110 to varying degrees.

At a leading edge 200 of the cage 100 is positioned a beveled tip 210, with the beveled tip desirably including a first beveled surface pair 220 and a second beveled surface pair 230, these beveled surface pairs 220 and 230 desirably facilitate the insertion of the cage into the targeted anatomy. An opening 240 is also provided at the beveled tip, which can facilitate the placement of the cage 100 using a guidewire and/or other surgical tools, as desired.

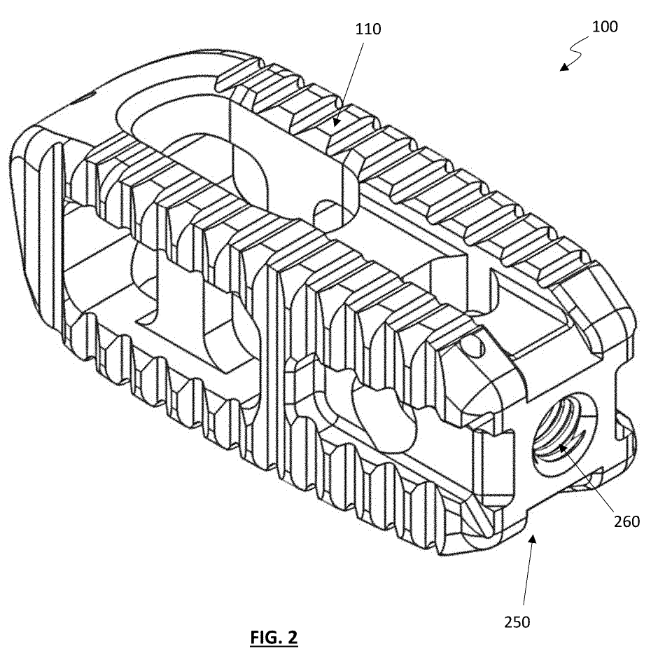

As best seen in FIG. 2, the cage 100 further includes a trailing edge 250 which desirably includes an internally threaded opening 260. In various embodiments, the internally threaded opening 260 can accommodate an externally threaded deployment tool (not shown), which engages with the cage and is used to place the cage in a desired position and orientation in the targeted anatomy, with the deployment tool unthreaded and removed when the cage 100 is in a desired location. Where repositioning and/or removal of the cage may be desirous, the deployment tool may be rethreaded into the cage as necessary.

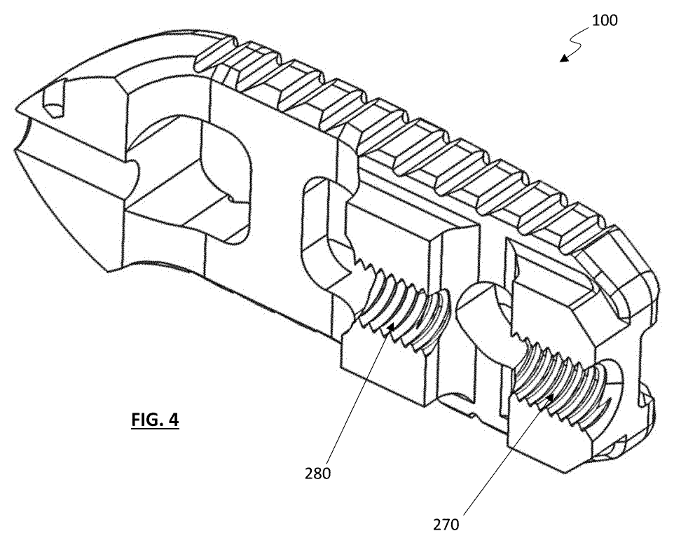

Referring now to FIG. 4, which depicts a cross-sectional view of the cage 100, it can be seen that the internally threaded opening 260 can extend to a second threaded section 270, which can greatly increase the rigidity, strength and/or durability of the cage 100 during insertion of the device.

If desired, the central void 180 will comprise one or more graft chamber and/or channels that may be filled with a radiolucent material such as tissue grafts. For instance, the graft chamber(s) may be packed with bone graft, and the one or more channels may be filled with, for example, putty style graft material. Bone graft material may facilitate bone and tissue ingrowth in and through the cage. Accordingly, bone graft may promote fusion, i.e., the joining of two or more vertebrae.

Referring back to FIG. 1, the cage 100 desirably includes a primary height H_{P}, and a secondary height H_{S}, wherein H_{P} and H_{S} are different heights. For example, in one embodiment of a cage implant H_{P} might be approximately 11 millimeters, with H_{S} being 8 millimeters. During use, such as depicted in FIGS. 3A, 3B and 3C, the different primary and secondary heights of the cage 100 can be utilized to accommodate different anatomical situations, as desired by the surgeon.

In contrast, prior art cages typically have a geometric surface texturing only on the superior and inferior surfaces, with typical implant offerings being a fixed height and width cage. For these systems, it is necessary to have multiple cages available to satisfy the require implant height, which can be further exasperated by the need for multiple cages when treating single or multiple levels. In contrast, with a dual-position cage such as disclosed herein, a single cage can accommodate multiple anatomical spacing requirements, with the user having the option to select the “a” side or “b” side as the base of the cage. This helps eliminate the need to interchange one cage to another into an insertion tool. This also helps eliminate the amount of inventory needed for a case, freeing up the back-table “real estate” in an operating room.

In various embodiments, a kit of implant cages can be provided, such as a kit containing lumbar cages having widths of 8, 9, 10 and 11 mm, heights of 5, 6, 7, 8, 9, 10, 11, 12, 13, 14, 15 and 16 mm (desirably excluding cages with the same width and height, as well as only having a single cage of each width/height or height/width combination), in lengths of 22, 24, 26, 28, 30, 32 mm, which significantly reduces the number of implants necessary for both the end user and the provider.

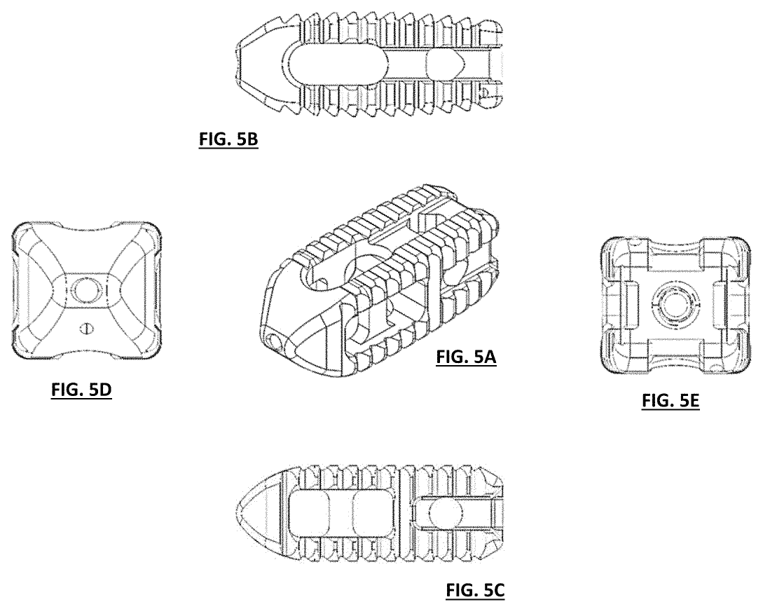

In various embodiments, including those of FIGS. 5A through 5E, the various superior/inferior and medial/lateral surfaces of the cage can be particularized for use in contact with the targeted anatomical structures, such as in intimate contact with the endplates of the superior and/or inferior vertebral bodies of an anatomical region of interest. In such cases, one or more of the surfaces may comprise a flattened and/or concave surface, which will desirably improve stability between the surface and the adjacent anatomy. In alternative embodiments, however, one or more of the surfaces may comprise more complex features, including curved and/or convex surface features, especially where a plurality of cages may be implanted into a single vertebral level.

In various alternative embodiments, the Primary and/or Secondary surface pairs of a single implant may comprise different surfaces, and/or the surfaces of a given surface pair may differ within the pair, which may allow the implant to be “flipped over” and/or other rotated to accommodate a desired anatomical situation. In various embodiments, the surfaces may include a plurality of bone interface members, such as, for example, teeth, serrations, protrusions (e.g., triangular, pyramidal, conical, semi spherical, rectangular, cylindrical, diamond, elliptical, and/or irregular shapes, or the like). Desirably, the bone interface members can engage with the bony surface of vertebral bodies in or near the treated area. If desired, the bone interface members can be formed integrally with the surface and may vary in profile, distribution, size, and/or number. The configuration of the surfaces should desirably be sufficient to securely hold the cage in the treated area after surgery while the treated area heals and undergoes fusion.

In various other embodiments, the relative heights, surface angles and/or anterior/posterior angulation may be different between the Primary and Secondary pairs, as desired.

Various of the embodiments disclosed herein can be manufactured from a variety of materials and/or material combinations, including polymers, such as poly-ether-ether-ketone (PEEK), poly(methyl methacrylate), poly(ethylene terephthalate), poly(dimethylsiloxane), poly(tetrafluoroethylene), polyethylene, and/or polyurethane, as well as other materials used to form other biomedical implants, such as metals, including Titanium, Silver, Nitinol, Platinum, Copper, Cobalt/Chromium, and related alloys, for example.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or dearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cage for implanting in a disc space between an upper vertebral body and a lower vertebral body of a patient, the cage having a main body comprising:
an upper surface and an opposing lower surface on an outer surface of the main body, the upper and lower surfaces including a first set of surface features appropriate for placement against bone surfaces of the upper and lower vertebral bodies, the upper and lower surfaces separated by a first distance; and
a medial surface and an opposing lateral surface on the outer surface of the main body, the medial and lateral surfaces including a second set of surface features appropriate for placement against bone surfaces of the upper and lower vertebral bodies, the medial and lateral surfaces separated by a second distance;
wherein the first distance is different than the second distance.

2. The cage of claim 1, wherein the first set of surface features and the second set of surfaces features are identical.

3. The cage of claim 1, wherein the first set of surface features is different from the second set of surfaces features.

4. The cage of claim 1, wherein the main body includes at least one first opening extending completely through the main body and the upper and lower surfaces.

5. The cage of claim 4, wherein the main body further includes at least one second opening extending completely through the main body and the medial and lateral surfaces.

6. The cage of claim 4, wherein the at least one first opening is in fluid communication with the at least one second opening.

7. The cage of claim 1, wherein the first set of surface features comprises identical surface features on each of the upper and lower surfaces.

8. The cage of claim 1, wherein the second set of surface features comprises identical surface features on each of the medial and lateral surfaces.

9. The cage of claim 4, wherein the at least one first opening comprises a plurality of first openings extending completely through the main body and the upper and lower surfaces.

10. The cage of claim 9, wherein the main body further includes a plurality of second openings extending completely through the main body and the medial and lateral surfaces, the plurality of first openings being in fluid communication with the plurality of second openings.

11. The cage of claim 10 further including a first end and a second end, with an opening extending from the first end to the second end along a longitudinal axis of the main body, the opening in fluid communication with the first and second openings through the main body.

12. A cage for implanting between adjacent vertebral bodies of a patient, the cage comprising:
a main body having an outwardly facing upper surface and an outwardly lower surface, the upper and lower surfaces including a first set of surface features appropriate for placement against bone surfaces of the adjacent vertebral bodies, the upper and lower surfaces separated by a first average separation; and
the main body further having an outwardly facing medial surface and an outwardly facing lateral surface, the medial and lateral surfaces including a second set of surface features appropriate for placement against bone surfaces of the adjacent vertebral bodies, the medial and lateral surfaces separated by a second average separation;
wherein the first average separation is different than the second average separation.

13. The cage of claim 12, wherein each of the upper, lower, medial and lateral surfaces are generally planar.

14. The cage of claim 12, wherein each of the upper, lower, medial and lateral surfaces are generally concave.

15. The cage of claim 12, wherein each of the upper, lower, medial and lateral surfaces are generally convex.

16. The cage of claim 12, wherein the medial and lateral surfaces are rotated approximately 90 degrees along a longitudinal axis of the main body from the upper and lower surfaces.

17. The cage of claim 12, wherein at least one of the upper, lower, medial and lateral surfaces are inclined.

18. The cage of claim 12, wherein the main body includes at least one first opening extending completely through the main body and the upper and lower surfaces.

19. The cage of claim 18, wherein the main body further includes at least one second opening extending completely through the main body and the medial and lateral surfaces.

20. The cage of claim 19, wherein the at least one first opening is in fluid communication with the at least one second opening.
